(19) United States
(12) Patent Application Publication
(10) Pub. No.: US 2011/0270164 A1
(43) Pub. Date: Nov. 3, 2011

(54) Nursing extension device

(76) Inventor: Lori Beth Bane, Gulf Breeze, FL (US)

(21) Appl. No.: 13/068,030

(22) Filed: May 2, 2011

Related U.S. Application Data
(60) Provisional application No. 61/330,817, filed on May 3, 2010.

(51) Int. Cl. A61M 1/06

(52) U.S. Cl. 604/76

(57) ABSTRACT

The invention is a device for allowing breast-feeding of infants to occur from a short distance away without the steps involved in pumping and bottle-feeding mother's milk. An example of such use is while mother and infant are riding in a car remaining safely buckled into a car seat and seat belt, respectively. The one- or three-piece device connects mother to infant via flexible tubing with an open-centered, adhesive breast shield to collect milk at one end and an artificial nipple for suckling at the other end. The invention can be easily cleaned and sterilized for multiple uses. The device can be a simple, single unit or three simple easily detachable pieces to allow for even easier production and cleaning.

### CROSS-REFERENCE TO RELATED APPLICATIONS

This is an application for converting the provisional patent application No. 61/330,817 filed May 3, 2010 to a non-provisional patent.

### STATEMENT REGARDING FEDERALLY SPONSORED RESEARCH OR DEVELOPMENT

This invention was not made under federally sponsored research or development.

### REFERENCE TO SEQUENCE LISTING, A TABLE, OR A COMPUTER PROGRAM LISTING COMPACT DISC APPENDIX

Not applicable.

### BACKGROUND OF THE INVENTION

The nursing extension device pertains generally to the field of infant feeding by nursing mothers and, more specifically, to a nursing extension device wherein a mother can feed her infant breast milk while a short distance away.

The American Academy of Pediatrics has recommended that mothers breastfeed exclusively for the first six months of a baby's life and that breastfeeding continue through the first year if mother and baby desire. Breastfeeding is a healthy, low-cost option for mothers and their babies. Support for breastfeeding as well as acceptance of breastfeeding in public has been increasing.

Young infants may need to eat/nurse as frequently as every two hours. There are certain times when this can be problematic for mothers who are breastfeeding, such as when they are riding in the car.

The mother can use a breast pump to feed the baby using a bottle, but this two-step process can be cumbersome to complete in a moving vehicle or in other situations where space is limited. An alternative is for the driver to stop the vehicle so mother and child can unbuckle their safety belts to move into a comfortable position for nursing. This is inconvenient and can increase travel time. An effective solution is needed.

The object of the present invention is to provide a comfortable, safe, economical and easy-to-use device to allow mother and baby to nurse in an environment such as riding in a car.

### BRIEF SUMMARY OF THE INVENTION

The nursing extension device of the present invention can be used to simplify breastfeeding while traveling in a vehicle. This unique device allows mother and child to remain safely buckled in their seats while nursing. The adhesive pad of the extension can be placed on the mother's breast, centered over the nipple, and extends for the baby to reach the nipple-shaped end from his or her rear- or forward-facing car seat. Suction created by the baby on the nipple-shaped end can deliver the mother's milk to the baby. Mother and baby can remain comfortably and safely strapped in without having to stop the vehicle or attempt to use a breast pump while the vehicle is in motion. Nursing mothers will appreciate the convenience and functionality this product offers.

The objects and features of the invention will be described in detail with reference to the accompanying drawings.

### BRIEF DESCRIPTION OF THE SEVERAL VIEWS OF THE DRAWING

FIG. 1 is a side view showing an embodiment of the invention.

FIG. 2 is a perspective view of the artificial nipple end of FIG. 1.

FIG. 3 is a perspective view of the breast-shield end of FIG. 1.

### DETAILED DESCRIPTION OF THE INVENTION

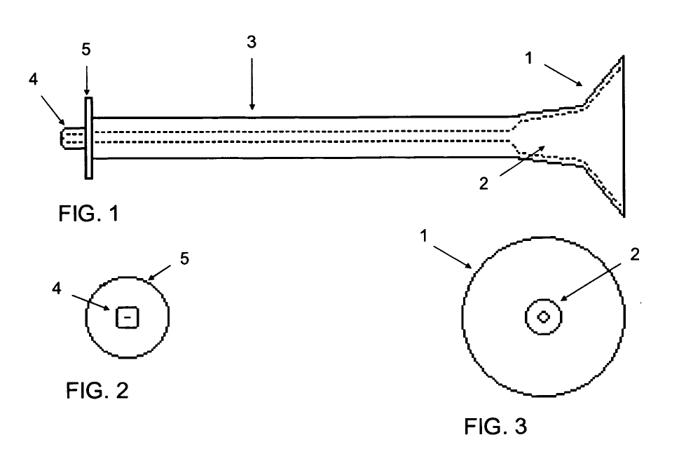

Referring to FIG. 1, the nursing extension device can be used to assist a breastfeeding mother to nurse her baby. This device can be made of BPA-free silicone or other similar soft, supple materials. One end 1 may be enlarged and slightly dome-shaped for attaching to the mother's breast, centered over the mother's nipple. This breast-shield end 1 can be made of a dome-shaped adhesive pad. The opening 2 to a tube 3 centers over the mother's nipple. The device can extend as tubing 3 to the opposite end 4, which can be perforated like a bottle nipple, for the infant to suckle. Part of the tubing 3 near the nipple-shaped end 4 can be enlarged in diameter 5 to substantially prevent the artificial nipple 4 from going too far into the infant's mouth. The present invention can be designed as a one- or three-piece unit enabling it to be conveniently cleaned, sanitized, and reused.

The three-piece unit could have the breast-shield 1 and nipple-shaped 4 ends detach from the tubing 3 by twisting and pulling differing diameters of tubing segments while retaining proper seal when re-attached. The device can be made of BPA-free silicone or other food-safe materials in various ways without limiting the scope of the present invention. Colors, methods of construction, and other specifications may vary upon manufacturing.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplifying embodiment was chosen and described in order to best explain the principles of the present invention and its practical application to thereby enable others skilled in the art to best utilize the present invention and various embodiments with various modifications as are suited to the particular use contemplated.

What is claimed is:

1. A single-piece mobile breast-feeding device comprised of a conically-shaped breast shield that connects to a perforated nipple-shaped end via a flexible tube that is rigid enough to prevent collapse when suction from the nipple-end is applied.

2. The single-piece mobile breast-feeding device of claim 1, wherein the breast shield end is made of supple, adhesive materials that can be washed, sterilized, and re-used many times while retaining its adhesive properties.

3. The single-piece mobile breast-feeding device of claim 1, wherein the system is made of BPA-free, food-safe materials.

4. The single-piece mobile breast-feeding device of claim 1, wherein the nipple-shaped end has a flared ring to prevent the nipple from going too far into the suckling infant's mouth.

5. The single-piece mobile breast-feeding device of claim 1, wherein the breast shield opening-to-tubing can come in various sizes to accommodate varying nipple sizes of the mother.

6. The single-piece mobile breast-feeding device of claim 1, wherein the nipple-shaped end can come in various sizes and shapes to accommodate the range of mouth sizes necessary for nursing infants to toddlers as well as personal suckling preferences.

7. The single-piece mobile breast-feeding device of claim 1, wherein the length of the tubing connecting the breast shield and nipple-shaped ends varies to accommodate different distances from the infant in various child safety restraints.

8. The single-piece mobile breast-feeding device of claim 1, wherein the materials are suitable for being washed in the dishwasher or in a micro steam bag for sterilization purposes.

9. A three-piece mobile breast-feeding device comprised of a conically-shaped breast shield and perforated artificial nipple that can be attached to and detached from opposite ends of a flexible tube by overlapping, sealing tubes of slightly different inner and outer diameters to facilitate cleaning and matching variations in breast-shield and nipple-shaped ends.

10. The three-piece mobile breast-feeding device of claim 9, wherein the breast shield attachment is made of supple, adhesive materials that can be washed, sterilized, and re-used many times while retaining its adhesive properties.

11. The three-piece mobile breast-feeding device of claim 9, wherein the system is made of BPA-free, food-safe materials.

12. The three-piece mobile breast-feeding device of claim 9, wherein the nipple-shaped attachment has a flared ring to prevent the nipple from going too far into the suckling infant's mouth.

13. The three-piece mobile breast-feeding device of claim 9, wherein the breast shield opening-to-tubing can come in various sizes to accommodate varying nipple sizes of the mother.

14. The three-piece mobile breast-feeding device of claim 9, wherein the nipple-shaped end can come in various sizes and shapes to accommodate the range of mouth sizes necessary for nursing infants to toddlers as well as personal suckling preferences.

15. The three-piece mobile breast-feeding device of claim 9, wherein the length of the tubing attaching the breast-shield and nipple-shaped ends varies to accommodate different distances from the infant in various child safety restraints.

16. The three-piece mobile breast-feeding device of claim 9, wherein the materials are suitable for being washed in the dishwasher or in a micro steam bag for sterilization purposes.
